(19) United States
(12) Patent Application Publication
(10) Pub. No.: US 2004/0241150 A1
(43) Pub. Date: Dec. 2, 2004

(54) Defined competitive exclusion cultures for food borne pathogens

(76) Inventors: Billy M. Hargis, Fayetteville, AR (US); Daniel J. Donoghue, Springdale, AR (US); Ann M. Donoghue, Springdale, AR (US); Noel K. Neighbor, Goshen, AR (US); Lisa R. Bielke, Springdale, AR (US); Lisa A. Newberry, Springdale, AR (US); Guillermo I. Tellez, Fayetteville, AR (US); Gerardo Nava, Fayetteville, AR (US)

Correspondence Address: Ray F Cox Jr, Wright Lindsey & Jennings, Suite 2300, 200 West Capitol Avenue, Little Rock, AR, 72201, US

(21) Appl. No.: 10/493,091

(22) PCT Filed: Dec. 19, 2002
(86) PCT No.: PCT/US02/41459

(51) Int. Cl. C12N 1/20; A61K 45/00

(52) U.S. Cl. 424/93.45; 435/252.9

(57) ABSTRACT

The invention comprises a method of screening cecal microflora for in vitro competition against select enteric pathogens, as well as the characterization of specific cecal microflora. The invention also comprises using the characterized competitive exclusion culture against select enteric pathogens in poultry, porcine, and ruminant, and humans. Selection of competitive isolates was achieved by combining Salmonella enteritidis (SE), adult broiler cecal microflora, and an indicator broth, which was then placed in a microtiter plate. Potential isolates were then challenged individually against Salmonella enteritidis. The ability of the selective isolates was determined by qualitative evaluation of Salmonella growth on brilliant green agar after the challenge. Isolates analyzed included but are not limited to; Bacillus species, Staphylococcus species, Escherichia species, Citrobacter species, Enterococcus species, Proteus species, Enterobacter species, Salmonella species, and Klebsiella species. Each isolate from genera with potential pathogens was further safety-tested by intraperitoneal injection or airsac injection and evaluated for morbidity, mortality, and lesions. We demonstrate that the incidence of SE recovery was markedly reduced by treatment with nine selected isolates which were individually amplified, under aerobic conditions, and then re-combined prior to administration.

### TECHNICAL FIELD

The intentional early colonization of the intestinal tract with beneficial microflora, known as competitive exclusion, has been shown to successfully protect poultry from selected enteric pathogens. The present invention relates to a defined, inexpensive, and air tolerant culture, as well as the methodology to obtain and store a competitive exclusion culture.

### BACKGROUND ART

Many of the more than 200 pathogenic serovars of the genus Salmonella are able to colonize the gastrointestinal tract of poultry (Gast, 1997). Neonatal chicks are susceptible to infection by very low numbers of Salmonella and Campylobacter, with increasing resistance as the birds, and presumably, normal enteric microflora mature (Byrd et al., 1998, Young et al., 1999). While most paratyphoid Salmonella infections of poultry are subclinical, poultry products have been reported to provide an important vehicle for human infections. An estimated 1.4 million cases of food-borne salmonellosis occur annually, with the total combined costs associated with medical care and lost productivity in the United States estimated at up to $3.5 billion annually (United States Department of Agriculture, 1995).

Competitive exclusion (CE), first described by Nurmi and Rantala (1973), has been an effective method of control for salmonellosis in commercial poultry flocks. Numerous mixed and undefined cecal cultures have been demonstrated to provide marked protection against Salmonella infection (Pivnick and Nurmi, 1982; Mead and Impey, 1986; Bailey, 1987; Stavric and D'aoust, 1993). Commercially available PREEMPT™ is a nearly-defined culture that is continuously produced from the same seed stock, arguably reducing the risk of reintroduction of pathogens (Corrier et al., 1995; Nisbet et al., 1996). However, other existing cultures are periodically amplified or passaged in specific pathogen free birds (Snoeyenbos et al., 1979; Nurmi et al., 1987). While these cultures are effective (Impey et al., 1987; Schneitz and Nuotio, 1992; Nisbet et al., 1996), a completely defined culture that is continuously derived from a single defined group of bacteria will be an inexpensive, safe alternative. To date, most investigators have argued that complex microflora may be required for effective CE to allow redundancy for adaptation to variable microenvironments within the gastrointestinal tract (Corrier et al., 1995; Mead 2000).

Additionally, attempts to create defined and effective CE cultures have been previously thwarted by the assumptions that selective media for many enteric microflora are not available, only approximately one quarter of the intestinal microflora have been characterized, and valid in vitro selection criteria have not been demonstrated (Mead, 2000).

The present invention comprises a method to select for individual enteric bacteria capable of inhibiting growth of specific enteric pathogens in vitro. In addition, the invention pertains to a method of selecting a combination of the selected oxygen- and freeze-tolerant bacteria to protect poultry and livestock from infection following challenge with an enteric pathogen. While the invention is not limited to the presently-selected beneficial bacteria, a specific group of bacteria which have been demonstrated to have efficacy in vivo are also claimed.

### DISCLOSURE OF THE INVENTION

Abbreviations and Definitions

The following definitions and methods are provided to better define the materials and methods disclosed herein and to guide those of ordinary skill in the art in the practice of the invention.

BGA: brilliant green agar

CE: competitive exclusion

CFU: colony forming units

NA: naladixic acid

NO: novobiacin

OD: optical density

SE: Salmonella enteritidis

TSA: tryptic soy agar

TSB: tryptic soy broth

### BRIEF DESCRIPTION OF THE DRAWINGS

These and other features, objects, and advantages, of the present invention will become better understood from a consideration of the following detailed description and accompanying drawings.

The combined bacteria (Table 1) were initially evaluated in vivo following growth of all the bacteria in a single batch culture (FIGS. 1-4).

FIG. 1. CE culture was grown as a combined culture for 12 hours in pre-reduced tryptic soy broth at 40 C in a modified anaerobic incubator, flushed with 6 volumes of CO₂. Optical density (625 nm) was determined at 2,4,6,8, and 12 hours. Early amplification of this combination culture is evident as early as 4 hours and reaches the plateau growth phase at approximately 8 hours.

FIG. 2. Comparison of 4 and 7 hour batch culture of competitive exclusion and Salmonella entgeritidis recovery in turkey poults. Specific combinations of bacterial isolates, described in Table 1, were initially tested at either 4 or 7 hours. The 4 hour culture was demonstrated to be more efficacious than the 7 hour culture at low concentrations, possibly due to diminution of bacterial diversity within the batch culture. CE culture was grown as a combined culture for 4 hour or 7 hour in pre-reduced tryptic soy broth at 40 C in a modified anaerobic incubator, flushed with 6 volumes of CO₂. Both cultures were adjusted to approximately the same optical density (625 nm) prior to dilutions, and colony-forming units were determined by plating serial dilutions on tryptic soy agar. Culture was diluted to appropriate dilution in 0.9% saline and administered to day-of-hatch poults via oral gavage before placement in floor pens. Control birds were administered 0.9% saline. All poults were challenged with 10² cfu Salmonella enteritidis two days after placement. Feed and water were provided ad libitum. Cecal tonsils were aseptically removed from poults at seven days after placement and conventionally enriched for Salmonella enteritidis. ^{a,b,c} bars with different superscripts are significantly different (p<0.05). ^{x,y} bars within treatment dose with different superscripts are significantly different (p<0.05).

FIG. 3. The effect of 4 hour batch culture of competitive exclusion on Salmonella enteritidis recovery in turkey poults. Appropriate dilutions (doses) of subsequent cultures using this batch amplification technique are highly efficacious in preventing Salmonella infection when harvested after 4 hour incubation. In this separate experiment, the culture was also demonstrated to be highly potent, with observed optimal efficacy when the culture was diluted 10,000- or 100,000-fold. CE culture was grown as a combined culture for 4 hours in pre-reduced tryptic soy broth at 40 C in a modified anaerobic incubator, flushed with 6 volumes of CO₂. Culture was diluted to appropriate dilution in 0.9% saline and administered to day-of-hatch poults via oral gavage before placement in floor pens. Control birds were administered 0.9% saline. All poults were challenged with 10² cfu Salmonella enteritidis two days after placement. Feed and water were provided ad libitum. Cecal tonsils were aseptically removed from poults at seven days after placement and conventionally enriched for Salmonella enteritidis. ^{a,b c} bars with different superscripts are significantly different (p<0.05).

FIG. 4. The effect of 4-hour batch culture of competitive exclusion on Salmonella enteritidis recovery in turkey poults. This batch amplification technique was highly efficacious in preventing Salmonella infection when harvested after 4 hours incubation. In this separate experiment, the culture was also demonstrated to be highly potent, with observed optimal efficacy when the culture was diluted 10,000-, 100,000-, or 1,000,000-fold. CE culture was grown as a combined culture for 4 hours in pre-reduced tryptic soy broth at 40 C in a modified anaerobic incubator, flushed with 6 volumes of CO₂. Culture was diluted to appropriate dilution in 0.9% saline and administered to day-of-hatch poults via oral gavage before placement in floor pens. Control birds were administered 0.9% saline. All poults were challenged with 10⁴ cfu Salmonella enteritidis two days after placement. Feed and water were provided ad libitum. Cecal tonsils were aseptically removed from poults at seven days after placement and conventionally enriched for Salmonella enteritidis. ^{a,b,c} bars with different superscripts are significantly different (p<0.05).

To reduce the potential cost of production and quality control, the ability to grow a select set of the beneficial bacteria as individual isolates, under aerobic conditions, and then re-combine the amplified isolates for administration was subsequently evaluated in separate experiments for turkeys and chickens (FIGS. 5-7). In these experiments, poults (FIGS. 5 and 6) or chicks (FIG. 7) were treated with serial dilutions of the combined nine bacterial isolates on the day-of-hatch by oral gavage and in the drinking water as described as below. Each of these methods of administration resulted in significant reduction of Salmonella infection following challenge with 10³ cfu Salmonella enteritidis 48 hours after hatch.

FIG. 5. Poults treated with serial dilutions of combined nine bacterial isolates on the day-of-hatch. Nine bacterial isolates (Table 2) selected from the original isolates identified in Table 1, and additional isolates described in Table 3. Nine CE isolates were grown individually in appropriate media for 12 hours at 37. Each isolate was concentrated by centrifugation at 3000 rpm for 15 min and reconstituted by adding the original volume of fresh tryptic soy broth or MRS. The isolates were combined and diluted to the appropriate concentration in 0.9% saline. Twenty poults per group were orally gavaged on the day-of-hatch. The treatment group receiving the −2 dilution received 1.68×10⁸ cfu per poult, and the −4, −6, and −8 groups received 100-fold dilutions respectively. The control group was gavaged with sterile saline. Poults gavaged with CE also received CE in the water for 4 consecutive days. The −2 group received 1.12×10⁵ cfu/mL in the water, and the other treatment groups also received 100-fold dilutions respectively. All poults were challenged with 2.5×10⁴ cfu of Salmonella enteritidis two days after initial treatment. Cecal tonsils were aseptically removed from poults 5 days after placement and conventionally enriched for Salmonella enteritidis. Bars with different superscripts are significantly different.

FIG. 6. Poults treated with serial dilutions of combined nine bacterial isolates on the day-of-hatch. Nine bacterial isolates (Table 2) selected from the original isolates identified in Table 1, and additional isolates described in Table 3. Nine CE isolates were grown individually in appropriate media for 12 hours at 37. Each isolate was concentrated by centrifugation at 3000 rpm for 15 min and reconstituted by adding the original volume of fresh tryptic soy broth or MRS. The isolates were combined and diluted to the appropriate concentration in 0.9% saline. Fifteen poults per group were orally gavaged on the day of hatch. The treatment group receiving the −4 dilution received 4.16×10⁵ cfu per poult, and the −5, and −6 groups received 10-fold dilutions respectively. The control group was gavaged with sterile saline. Poults gavaged with CE also received CE in the water for 4 consecutive days. The −4 group received 600 cfu/mL in the water, and the other treatment groups also received 10-fold dilutions respectively. All poults were challenged with 5.0×10³ cfu of Salmonella enteritidis two days after initial treatment. Cecal tonsils were aseptically removed from poults 5 days after placement and conventionally enriched for Salmonella enteritidis. Bars with different superscripts are significantly different.

FIG. 7. Chicks treated with serial dilutions of combined nine bacterial isolates on the day-of-hatch. Nine CE isolates were grown individually in appropriate media for 12 hours at 37. Each isolate was concentrated by centrifugation at 3000 rpm for 15 min and reconstituted by adding the original volume of fresh tryptic soy broth or MRS. The isolates were combined and diluted to the appropriate concentration in 0.9% saline. Twenty chicks per group were orally gavaged on the day of hatch. The treatment group receiving the −2 dilution received 1.09×10⁶ cfu per chick, and the −4, and −6 groups received 100-fold dilutions respectively. The control group was gavaged with sterile saline. Chicks gavaged with CE also received CE in the water for 4 consecutive days. All chicks were challenged with 1.0×10⁴ cfu of Salmonella enteritidis two days after initial treatment. Cecal tonsils were aseptically removed from chicks 5 days after placement and conventionally enriched for Salmonella enteritidis. Bars with different superscripts are significantly different.

FIG. 8. Effects of CE isolates on Campylobacter colonization. Similarly-selected cultures of candidate bacteria (yet unidentified) were selected on the basis of ability to exclude Campylobacter spp. in vitro. Subsequent ability to culture and recover Campylobacter jejuni was greatly reduced. Bacterial CE isolates were selected on the basis of in vitro competition with Campylobacter. A frozen culture of each isolate was inoculated into 10 mL Campylobacter Enrichment Medium (CEM) or Campylobacter Line Agar (CLA; Line et al., 2001) without antibiotics and allowed to grow for 6 to 8 hours at 37 C. Bacteria not exhibiting turbidity after 9 to 12 hours were transferred to 42 C and the culture used 3 to 6 hours later. The isolates were diluted with CEM or CLA and the OD adjusted to 0.15 which was estimated to contain 10⁸ CFU/mL. 100 μL of this diluted culture was added to 10 mL CEM, which corresponds to 10⁶ CFU/mL. Two more serial dilutions were done in CEM producing concentrations of 10⁵ & 10⁴ CFU/mL. All bacterial isolate tubes, were labeled and kept in the refrigerator till they were ready to be dispensed in 96 well plates. American Type Culture Collection (ATCC) 33291 Campylobacter jejuni and a wild type strain Campylobacter were used for challenge. Bars with different superscripts are significantly (p<0.05) different within experiments

### SUMMARY OF THE INVENTION

The intentional early colonization of the intestinal tract with beneficial microflora, known as competitive exclusion, has been shown to successfully protect poultry from selected enteric pathogens. While effective cultures have been produced and are available, an inexpensive, air tolerant and completely defined culture is needed. The invention comprises an in vitro competition assay to select for individual facultative anaerobes, of poultry enteric origin, that could exclude select enteric pathogens.

Selection of competitive isolates was achieved by combining Salmonella enteritidis, adult broiler cecal bacteria at low concentrations, and an indicator broth, which were co-incubated in a microtiter plate. Potential isolates were then challenged individually against Salmonella enteritidis. In this screening assay, culture wells in which Salmonella were able to grow changed to a dark black color, while culture wells with diminished or no Salmonella growth remained clear. Subsequent to the screening assay, the competitive ability of the selected isolates was determined by qualitative evaluation of Salmonella growth on brilliant green agar after the co-incubation. Isolates analyzed included but are not limited to; Bacillus species, Staphylococcus species, Escherichia species, Citrobacter species, Enterococcus species, Proteus species, Enterobacter species, Salmonella species, Klebsiella species, and lactic acid bacteria species. Each isolate from genera with potential pathogens was further safety-tested by intraperitoneal injection or airsac injection and evaluated for morbidity, mortality, and lesions. Groups of bacterial isolates selected in this fashion with demonstrated ability to exclude Salmonella infection in live poultry are detailed in Tables 1-3.

An embodiment of the invention comprises a method of screening cecal microflora for in vitro competition against select enteric pathogens.

Another embodiment of the invention characterizes specific cecal microflora.

In one embodiment of the invention is the characterization of the competitive exclusion culture for competition against select enteric pathogens.

Another embodiment of the invention is a method of storing the competitive exclusion culture.

In still another embodiment of the invention comprises using the characterized competitive exclusion culture against select enteric pathogens in poultry species.

In another embodiment of the invention comprises using the characterized competitive exclusion culture against select enteric pathogens in porcine species.

In one embodiment of the invention comprises using the characterized competitive exclusion culture against select enteric pathogens in ruminant species.

In one embodiment of the invention comprises using the characterized competitive exclusion culture against select enteric pathogens in humans.

### BEST MODE FOR CARRYING OUT THE INVENTION

With reference to the above-described Figs. the preferred embodiment of the present invention may be described.

Screening of cecal microflora for in vitro competition against Salmonella resulted in an effective defined competitive exclusion culture. Furthermore, we demonstrate that a relatively simple culture, containing only oxygen-tolerant bacteria, can indeed protect poultry when administered at the appropriate dose. Such a batch-produced culture offers advantages in terms of production cost, ability to easily control or validate quality of product, and reduce concerns with regard to unknown pathogens.

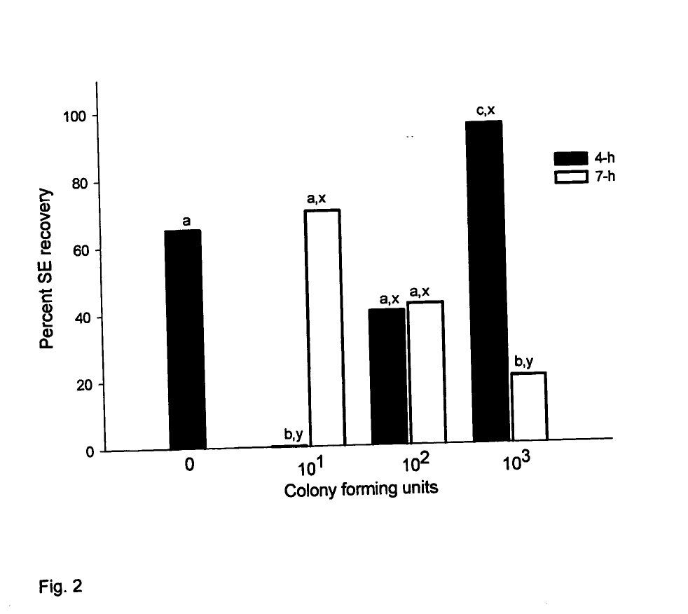

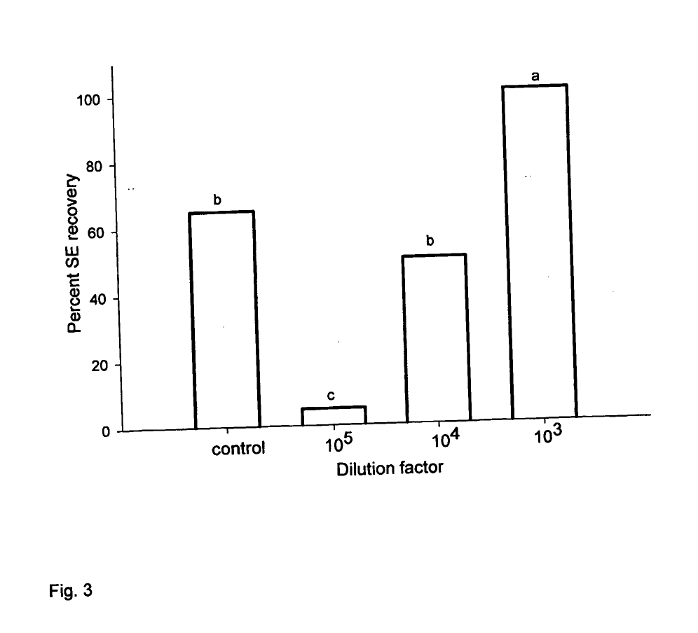

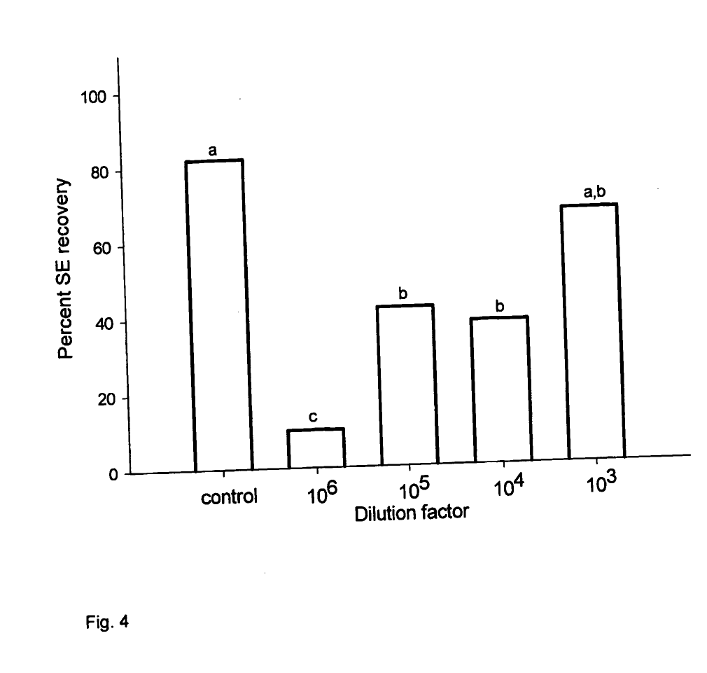

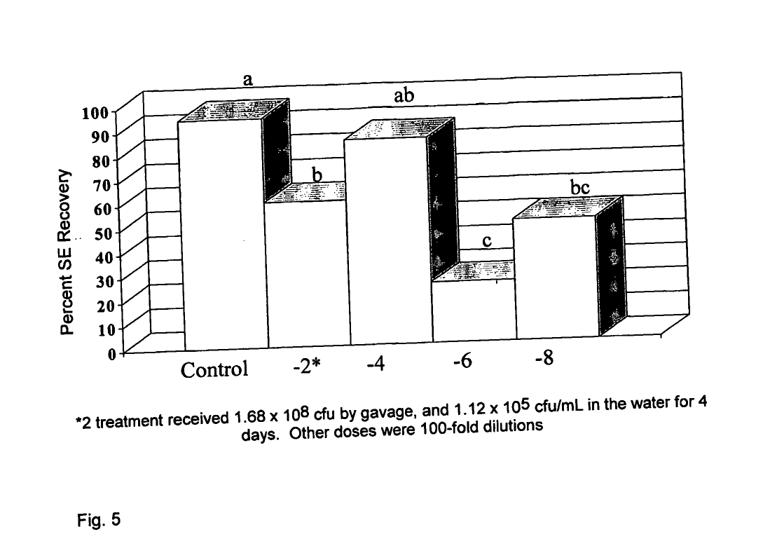

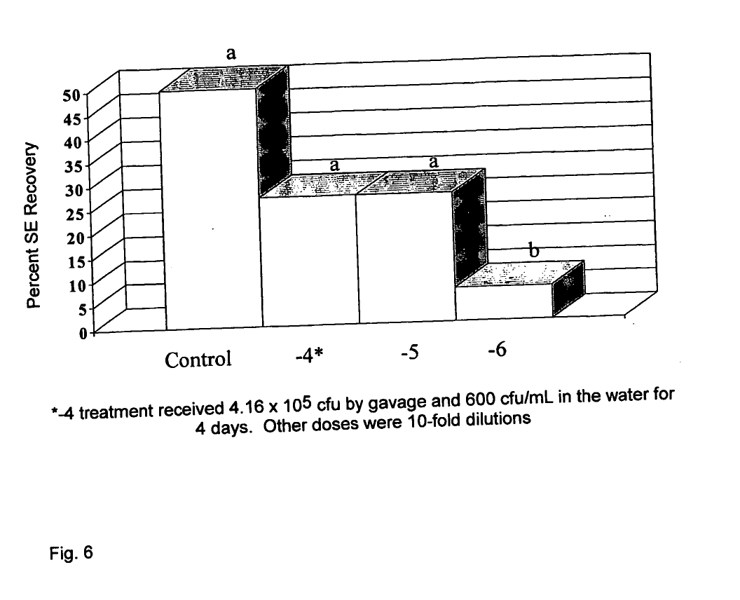

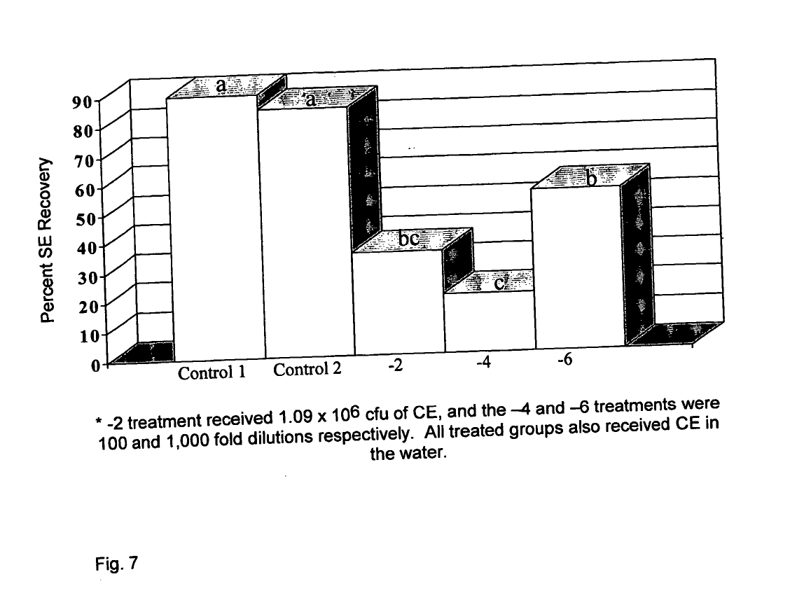

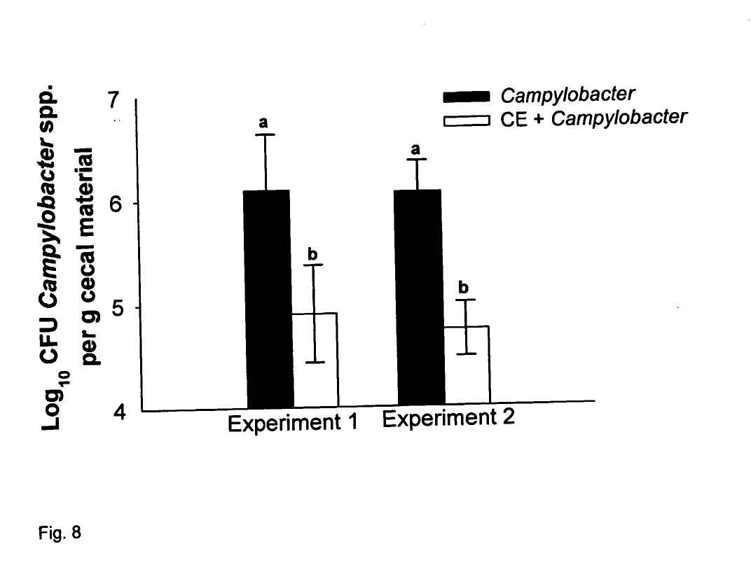

As shown in the accompanying Figs., the incidence of SE recovery was markedly reduced by treatment with either the original combined culture (Table 1) amplified together as a batch culture for 4 hours (FIGS. 2-4) or when nine selected isolates were individually amplified, under aerobic conditions, and then re-combined prior to administration to turkeys (FIGS. 5 and 6) or chickens (FIG. 7).

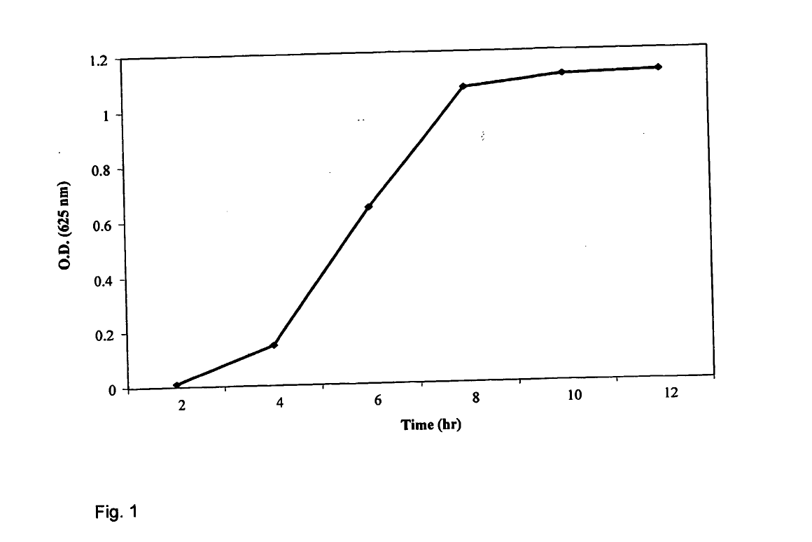

TABLE 1
Identification of Poultry Enteric Isolates Included in
Competitive Exclusion Culture as
Described in FIGS. 1-4.

| | | Arkansas |
|---|---|---|
| | | State Diagnostic |
| | | Laboratory |
| Isolate # | PHL Identification¹ | Identification² |
| 1 | Gram positive | Bacillus spp. |
| 2 | Gram positive | Staphylococcus xylosus |
| 3 | Escherichia coli | Escherichia coli |
| 4 | Klebsiella oxytoca | Klebsiella oxytoca |
| 5 | Citrobacter fruendii | Escherichia coli |
| 6 | Citrobacter fruendii | Escherichia coli |
| 7 | Gram positive | Staphylococcus spp. |
| 8 | Escherichia coli | Escherichia coli |
| 9 | Citrobacter fruendii | Citrobacter fruendii |
| 10 | Klebsiella oxytoca | Klebsiella oxytoca |
| 11 | Citrobacter fruendii | Escherichia coli |
| 12 | Escherichia coli | Escherichia coli |
| 13 | Escherichia coli | Escherichia coli |
| 14 | Citrobacter fruendii | Citrobacter fruendii |
| 15 | Citrobacter fruendii | Escherichia coli |
| 16 | Citrobacter fruendii | Escherichia coli |
| 17 | Citrobacter fruendii | Escherichia coli |
| 18 | Citrobacter fruendii | Escherichia coli |
| 19 | Citrobacter fruendii | Enterobacter cloacae |
| 20 | Gram positive | Enterococcus faecium |
| 21 | Escherichia coli | Escherichia coli |
| 22 | Escherichia coli | Escherichia coli |
| 23 | Escherichia coli | Escherichia coli |
| 24 | Escherichia coli | Escherichia coli |

¹Isolates identified using the API system at the University of Arkansas JKS Poultry Health Research Laboratory.
²Isolates identified at the Arkansas State Diagnostic Laboratory in Springdale, AR.

The present invention represents combinations of bacterial isolates retained for in vivo testing, and these isolates were selected through screening approximately 4-8×10⁶ cecal bacteria. The presently-selected isolates are described in Tables 1 and 2. In contrast to some previous speculations regarding the necessity of complex CE cultures for prophylactic efficacy (Corrier et al., 1995; Mead, 2000), the efficacy of this CE culture in protecting poults from Salmonella infection, when administered at the appropriate dose, indicates simple cultures can provide protection.

TABLE 2
Nine Selected Bacteria Included in Limited CE Culture
as Described in FIGS. 5 and 6.

| | | Arkansas |
|---|---|---|
| | | State Diagnostic |
| | | Laboratory |
| Identification # | PHL Identification/Description | Identification³ |
| 3 | Escherichia coli¹ | Escherichia coli |
| 8 | Escherichia coli¹ | Escherichia coli |
| 10 | Klebsiella oxytoca¹ | Klebsiella oxytoca |
| 12 | Escherichia coli¹ | Escherichia coli |
| 15 | Citrobacter fruendii¹ | Escherichia coli |
| 18 | Citrobacter fruendii¹ | Escherichia coli |
| 24 | Escherichia coli¹ | Escherichia coli |
| 27 | Weissella confusa² | |
| 35 | Gram positive cocci | |
| | clusters, isolated | |
| | from ceca - non typed | |

¹Isolates identified using API system at Poultry Health Research Laboratory.
²Isolates identified using BIOLOG system at University of Arkansas Center of Excellence for Poultry Science.
³Isolates identified at the Arkansas State Diagnostic Laboratory in Springdale, AR.

The invention pertains to a simple and defined CE cultures as well as to in vitro selection of such microflora. We also describe the negative effects of high doses of CE cultures, which have not been reported in previous studies with other cultures (Nurmi and Rantala, 1973; Schneitz and Nuotio, 1992; Corrier et al., 1995). The negative effects observed with the present CE combination were observed at doses at least 10 to 100-fold higher than those found to be effective (FIGS. 2-4). These negative effects of high doses of culture were only observed when the selected bacteria were grown under batch conditions. Negative effects of higher doses of bacteria (lower dilutions) were not observed when selected bacteria were grown as individual isolates (FIGS. 5-7).

TABLE 3
Identification/Description of Lactic Acid-Producing Bacteria
which Compete with Salmonella in vitro

| Isolate # | Identification/Description¹ |
|---|---|
| 25 | Gram positive, cocci clusters, |
| | isolated from ceca |
| 26 | Clostridium clostridiiforme |
| 27 | Weissella confusa |
| 28 | Lactobacillus hamsteri |
| 29 | Weissella confusa |
| 30 | Gram positive, bacillus isolated from |
| | duodenum - not identified |
| 31 | Weisella paramesenteroides |
| 32 | Gram positive, coccobacillus, |
| | isolated from ileum - not identified |
| 33 | Lactobacillus salivarius |
| 34 | Lactobacillus salivarius |
| 35 | Gram positive cocci clusters, isolated |
| | from ceca - not identified |

¹Isolates identified using BIOLOG system at the University of Arkansas Center of Excellence for Poultry Science.

### EXAMPLES

Salmonella Source

A primary poultry isolate of Salmonella enteritidis (SE), phage type 13A, was originally obtained from the National Veterinary Services Laboratory (Ames, Iowa). This isolate was selected for resistance to nalidixic acid (NA). For these experiments, Salmonella was grown in Tryptic Soy Broth (TSB) for approximately 8 hours. The cells were washed three times with 0.9% sterile saline by centrifugation (3000×g) and the approximate concentration of the stock solution was determined spectrophotometrically. The stock solution was serially diluted and confirmed by colony counts of three replicate samples (0.1 mL/replicate), spread plated on brilliant green agar (BGA) plates containing 25 μg/mL novobiocin (NO) and 20 μg/mL NA. The cfu of Salmonella determined by spread plating were reported as the concentration of Salmonella in cfu/mL for in vitro experiments and total cfus for in vivo challenge experiments. Salmonella recovery procedures have been previously described by our laboratory (Tellez et al., 1993).

In vitro Selection of Cecal Microflora

Ceca were aseptically removed from 46 healthy adult chickens housed at the University of Arkansas poultry farm and placed in sterile sample bags. Ceca were immediately frozen in liquid nitrogen for 1-3 hours and stored overnight at −80 C. Four grams of each sample was thawed and individually diluted 10-fold weight to volume in TSB. The final dilution of each sample was then added to the wells of a sterile 96-well microtiter plate (80 μL/well). Eighty μL of TSB containing SE at concentrations of 10³, 10⁴, or 10⁵ cfu/mL was added to the wells of the microtiter plates containing the cecal samples so that each well was inoculated with 3.3×10², 10³, or 10⁴ cfu of SE. Ferric ammonium citrate at a concentration of 2.75 g/100 mL TSB (10.1 mg/mL final concentration per well) and sodium thiosulfate at a concentration of 0.040 g/100 mL TSB (0.15 mg/mL final concentration per well) were used as indicators of SE growth and added at 80 μL/well.

Microtiter plates were incubated overnight in a bacteriological incubator at 37 C. Plates were then qualitatively examined for the presence or absence of black precipitate consistent with Salmonella growth (DIFCO Laboratories, 1984). Using a sterile loop, samples from the wells without black precipitate (no color change) were streaked for isolation on BGA and Tryptic Soy Agar (TSA) and incubated overnight in a bacteriological incubator at 37 C. All resulting non-Salmonella colonies were reisolated on TSA to ensure purity. A single, isolated colony was grown in 10 mL TSB for 8 hours, or until the culture was turbid. Sterile glycerol was then added to the culture in TSB and the suspension was aliquoted and stored at −80 C.

Continued screening began with growing each cecal isolate for eight hours in TSB in a bacteriological incubator at 37 C. Twenty-five μL of each cecal isolate was combined with 3.3×10³ cfu SE and 215 μL TSB in 96-well microtiter plates, in duplicate with supplemented medium as described above. Control plates consisted of the same arrangement of cecal isolates without SE. Paired plates were incubated in a bacteriological incubator at 37 C, or a modified anaerobic incubator, flushed with eight volumes of CO₂ prior to incubation at 40 C. To ensure the selection of facultative anaerobes, the optical density (625 nm) of the control plates incubated in the modified anaerobic chamber was determined. Subsequent to incubation of the microtiter plates, each well of the microtiter plates containing cecal isolates and SE were streaked on BGA that contained antibiotics to which the SE was resistant. After overnight incubation at 37 C, the BGA plates were qualitatively analyzed for the ability of the cecal isolates to inhibit SE growth as described above. All cecal isolates able to inhibit or reduce SE growth were selected for further study. Cecal isolates with the ability to reduce Salmonella, in vitro, were tested for their ability to also inhibit Campylobacter, in vitro.

All selected cecal isolates were identified in our laboratory using API strips and at the Arkansas State Diagnostic Laboratory (Springdale, Ariz.). All potential pathogens including genera of Escherichia, Klebsiella, and Proteus, were safety tested in broilers and turkeys (5 per tested isolate for each route of administration) by intraperitoneal, air sac, and subcutaneous injection. Inoculated birds were examined for morbidity, mortality, and lesions. All bacteria that caused any evidence of disease were removed as candidates from the culture. The remaining individual cecal isolates included in the culture were aliquoted and stored with 30% glycerol at −80 C

Additional isolates of lactic acid-produding bacteria (See Table 3) were evaluated individually for ability to compete with Salmonella enteritidis as described above with the following modifications. These isolates were isolated from the intestinal contents of ten healthy four-week-old turkeys. The entire intestinal tract was aseptically removed from each turkey, and the contents were collected individually from four regions of the gastrointestinal tract: the crop, duodenum, ileum, and cecae. From each region sampled, the contents from the ten turkeys were pooled in a sterile flask. Additionally, each gastrointestinal region sampled was rinsed with sterile saline and gently massaged in order to remove bacteria attached to the walls of the gastrointestinal tract. The saline rinse was collected and pooled separately from the contents collected. Serial ten-fold dilutions of all samples were performed, and the dilutions were spread plated on Mann-Rogosa-Sharpe agar, which is selective for lactic acid producing bacteria. Plates were incubated for 24 hours at 37 C. Resulting colonies exhibiting different morphology were isolated and serially plated at least 3 times to obtain a purified culture. Isolated colonies were then assayed for inhibition of Salmonella on Mann-Rogosa-Sharpe agar. Briefly, the isolated lactic acid bacteria were individually inoculated on the center of the agar and allowed to incubate for 24 hours at 37 C. Following incubation, 10⁷ cfu of Salmonella enteritidis PT 13A in 2 mL of soft agar was poured onto the plate, covering the original hard agar plate. The plate was allowed to incubate again for 24 hours at 37 C. Lactic acid bacteria that produced a zone of inhibition (clear zone around the lactic acid colony where Salmonella enteritidis PT 13A did not grow) were then further identified using the Biolog identification system (Table 3). Selected isolates were further characterized in combination with other bacteria (described in Table 1) for in vivo challenge trials as described in FIGS. 5-7.

Screening For in vitro Efficacy Against Campylobacter:

A frozen culture of each isolate was inoculated into 10 mL Campylobacter Enrichment Medium (CEM) or Campylobacter Line Agar (CLA; Line et al, 2001) without antibiotics and allowed to grow for 6 to 8 hours at 37 C. Bacteria not exhibiting turbidity after 9 to 12 hours were transferred to 42 C and the culture used 3 to 6 hours later. The isolates were diluted with CEM or CLA and the OD adjusted to 0.15 which was estimated to contain 10⁸ CFU/mL. 100 μL of this diluted culture was added to 10 mL CEM, which corresponds to 10⁶ CFU/mL. Two more serial dilutions were done in CEM producing concentrations of 10⁵ & 10⁴ CFU/mL. All bacterial isolate tubes, were labeled and kept in the refrigerator till they were ready to be dispensed in 96 well plates. American Type Culture Collection (ATCC) 33291 Campylobacter jejuni and a wild type strain Campylobacter were used for challenge.

CE Culture

A single aliquot of the combine culture containing the 24 selected isolates was thawed and grown in 500 mL of pre-reduced TSB for either 4 or 7 hours in a modified anaerobic incubator, flushed with eight volumes of CO₂, at 40 C. The culture was then serially diluted with 0.9% saline 10⁵X for administration to poults.

Poults

Commercial cross, British United Turkeys of America, poults were obtained on the day-of-hatch and orally gavaged with the appropriate culture before placement in floor pens. Treatments consisted of 20 poults per treatment group. Each pen was approximately 1.0 m² in area and the floor covered with clean softwood shavings. Poults were provided antibiotic-free feed, formulated to meet or exceed National Research Council (NRC) recommendations for critical nutrients for day-of-hatch poults (NRC, 1994) and water ad libitum. Poults were administered SE challenge at concentrations of 10² or 10⁴ cfu by oral gavage 48 hours after placement. At seven days after placement, poults were humanely killed, cecal tonsils aseptically removed, and enriched in 20 mL of tetrathionate broth. Cecal tonsils were incubated for 24 hours at 37 C, and streaked on BGA plates containing 25 μg/mL NO and 20 μg/mL NA. The plates were incubated for 24 hours at 37 C and examined for the presence of lactose-negative, NA-resistant Salmonella colonies. Selected lactose-negative, antibiotic-resistant colonies typical of Salmonella were further confirmed by serogrouping.

Experimental Design (In vivo Challenge)

For administration to poults or chicks, the CE culture was grown in 500 mL of pre-reduced TSB for either 4-hour or 7-hour at 40 C under reduced oxygen conditions as described above. The culture was then serially diluted in 0.9% saline at 10-fold increments. Poults were gavaged with 0.25 mL of culture or vehicle (controls) on day of hatch. In some experiments, selected bacteria were also included in the drinking water during the first four days of life (FIGS. 5-7). All poults were challenged with 10⁴ cfu or 10² cfu SE in 0.25 mL 0.9% saline 48-hours after placement by oral gavage. Seven days after placement, poults were humanely killed and cecal tonsils were aseptically removed. Cecal tonsils were enriched for SE recovery as described above.

Statistical Analysis

The chi-square test of independence was used to determine significant differences (P≦0.05) in Salmonella recovery between treatments within experiments testing all possible combinations as described in the figure (Zar, 1984).

### INDUSTRIAL APPLICABILITY

The invention pertains to competitive exclusion cultures that have been shown to successfully protect poultry or other species from selected enteric pathogens. The invention comprises an in vitro competition assay to select for individual facultative anaerobes that could exclude select enteric pathogens.

What is claimed is:

1. A method of preparing a composition of enteric microorganisms having the capability to competitively exclude a pathogenic microorganism from a species of animal, comprising the steps of:
a) obtaining a sample of enteric microorganisms from an animal of the species;
b) exposing said sample of enteric microorganisms to an aerobic environment for a sufficient period of time to produce a culture of microorganisms consisting essentially of aerobic and facultative anaerobic microorganisms.
c) exposing said sample to freezing and thawing temperatures for a sufficient period of time to produce a culture of microorganisms substantially free of microorganisms that are sensitive to freezing and thawing;
d) culturing each microorganism surviving from steps (b) and (c) in a separate culturing medium having an indicator for indicating the growth of the pathogenic microorganism;
e) inoculating each culturing medium with the pathogenic microorganism;
f) identifying each culturing medium in which growth of the pathogenic microorganism is substantially inhibited;
g) preparing a mixed culture of one or more microorganisms from culturing media in which growth of the pathogenic microorganism is substantially inhibited.

2. The method of claim 1 wherein the indicator of step (d) is a hydrogen sulfide solution.

3. The method of claim 1 wherein the indicator of step (d) is brilliant green agar.

4. The method of claim 1 wherein the indicator of step (d) is saffronin.

5. The method of claim 1 wherein the indicator of step (d) is methylene blue.

6. The method of claim 1 wherein the indicator of step (d) is ferric ammonium citrate.

7. The method of claim 1 wherein the indicator of step (d) is sodium thiosulfate.

8. The method of claim 1 wherein the animal is a poultry species.

9. The method of claim 1 wherein the animal is a porcine species.

10. The method of claim 1 wherein the animal is a ruminant species.

11. The method of claim 1 wherein the pathogenic microorganism is Salmonella species.

12. The method of claim 1 wherein the pathogenic microorganism is Campylobacter species.

13. The method of claim 1 wherein the pathogenic microorganism is Escherichia species.

14. The method of claim 1 wherein the pathogenic microorganism is Listeria species.

15. A composition produced by the method of claim 1.

16. A composition comprising populations of substantially pure bacteria exhibiting substantial resistance to aerobic environments, and freezing and thawing, in amounts effective for inhibiting pathogenic microorganism colonization in animals.

17. The bacteria of claim 16 selected from the group consisting of:
a) a Bacillus species,
b) a Staphylococcus species,
c) an Escherichia species,
d) a Kiebsiella species,
e) a Citrobacter species,
f) an Enterococcus species,
g) an Enterobacter species
h) a Weissella species,
i) a Lactobacilli species,
j) and a lactic acid bacterial species in an amount effective for inhibiting said pathogenic microorganism colonization in animals.

18. The bacteria of claim 17, wherein the species of Klebsiella is oxytoca.

19. The bacteria of claim 17, wherein the species of Citrobacter is fruendii.

20. The bacteria of claim 17, wherein the species of Escherichia is coli.

21. The composition of claim 16 further comprising a carrier.

22. The composition of claim 16 wherein said pathogenic microorganism is Salmonella species.

23. The composition of claim 16 wherein said pathogenic microorganism is Campylobacter species.

24. The composition of claim 16 wherein said pathogenic microorganism is Escherichia species.

25. The composition of claim 16 wherein said pathogenic microorganism is Listeria species.

26. The composition of claim 16 wherein the animal is a poultry species.

27. The composition of claim 16 wherein the animal is a porcine species.

28. The composition of claim 16 wherein the animal is a ruminant species.

29. A method of preparing a composition of enteric microorganisms having the capability to competitively exclude a pathogenic microorganism from a human, comprising the steps of:
a) obtaining a sample of enteric microorganisms from an animal of the species;
b) exposing said sample of enteric microorganisms to an aerobic environment for a sufficient period of time to produce a culture of microorganisms consisting essentially of aerobic and facultative anaerobic microorganisms;
c) exposing said sample to freezing and thawing temperatures for a sufficient period of time to produce a culture of microorganisms substantially free of microorganisms that are sensitive to freezing and thawing;
d) culturing each microorganism surviving from steps (b) and (c) in a separate culturing medium having an indicator for indicating the growth of the pathogenic microorganism;
e) inoculating each culturing medium with the pathogenic microorganism;
f) identifying each culturing medium in which growth of the pathogenic microorganism is substantially inhibited;
g) preparing a mixed culture of one or more microorganisms from culturing media in which growth of the pathogenic microorganism is substantially inhibited.

30. The method of claim 29 wherein the indicator of step (d) is a hydrogen sulfide solution.

31. The method of claim 29 wherein the indicator of step (d) is saffronin.

32. The method of claim 29 wherein the indicator of step (d) is methylene blue.

33. The method of claim 29 wherein the indicator of step (d) is ferric ammonium citrate.

34. The method of claim 29 wherein the indicator of step (d) is sodium thiosulfate.

35. The method of claim 29 wherein the pathogenic microorganism is Salmonella species.

36. The method of claim 29 wherein the pathogenic microorganism is Campylobacter species.

37. The method of claim 29 wherein the pathogenic microorganism is Escherichia species.

38. The method of claim 29 wherein the pathogenic microorganism is Listeria species.

39. A composition produced by the method of claim 29.

40. A composition comprising populations of substantially pure bacteria exhibiting substantial resistance to aerobic environments, and freezing and thawing, in amounts effective for inhibiting pathogenic microorganism colonization in humans.

41. The bacteria of claim 40 selected from the group consisting of:
a) a Bacillus species,
b) a Staphylococcus species,
c) an Escherichia species,
d) a Klebsiella species,
e) a Citrobacter species,
f) an Enterococcus species,
g) an Enterobacter species
h) a Weissella species,
i) a Lactobacilli species,
j) and a lactic acid bacterial species in an amount effective for inhibiting said pathogenic microorganism colonization in humans.

42. The composition of claim 40 further comprising a carrier.

43. The composition of claim 40 wherein said pathogenic microorganism is Salmonella species.

44. The composition of claim 40 wherein said pathogenic microorganism is Campylobacter species.

45. The composition of claim 40 wherein said pathogenic microorganism is Escherichia species.

46. The composition of claim 40 wherein said pathogenic microorganism is Listeria species.
